(19) United States
(12) Patent Application Publication
(10) Pub. No.: US 2022/0370785 A1
(43) Pub. Date: Nov. 24, 2022

(54) PUMP FOR MIMICKING PHYSIOLOGICAL BLOOD FLOW IN A PATIENT

(71) Applicant: Triphasic Cardiac Pump Pty Ltd, Nedlands (AU)

(72) Inventor: Albert Yoonsong Chong, Kallaroo (AU)

(21) Appl. No.: 17/761,759

(22) PCT Filed: Sep. 18, 2020
(86) PCT No.: PCT/AU2020/050995

§ 371 Date: Mar. 18, 2022

(30) Foreign Application Priority Data
Sep. 19, 2019 (AU) 2019903484

(51) Int. Cl. A61M 60/562; A61M 60/104; A61M 60/279; A61M 60/443; A61M 60/31; A61M 60/515

(52) U.S. Cl. CPC A61M 60/562; A61M 60/104; A61M 60/279; A61M 60/443; A61M 60/31; A61M 60/515

(57) ABSTRACT

A pump for mimicking physiological blood flow in a patient is disclosed. The pump works via compression and decompression of a tube, inducing a peristaltic flow within the tube. The compression may be effected by a linear actuator, or alternatively by a pivoting compression member. A one-way check valve ensures flow in a single direction.

### FIELD OF THE INVENTION

The present invention relates to a pump. The pump has been created for medical use, arranged to provide blood flow for use as part of a cardiopulmonary bypass profusion system or an extracorporeal membrane oxygenation system, or for use in therapeutic treatment of oxygen insufficiency in critical organs. Additionally, the pump can be arranged to function as an artificial heart. The pump is considered to have wider application, for instance in industrial applications where it can be programmed to dose a predetermined amount of fluid or semisolid material into a container at a desired filling profile.

### BACKGROUND TO THE INVENTION

Cardiac pumps used in surgery traditionally fall into two types: peristaltic and centrifugal. Both types of pump can be arranged to provide a consistent flow of blood at an appropriate pressure, with the pumps being adjustable to compensate for changes in flow rate or pressure requirements.

Traditional peristaltic and centrifugal pumps provide a pulsatile or continuous flow of blood. These have proved to be sufficient to maintain a patient through surgery.

It is notable, however, that the flow of blood provided by such cardiac pumps is notably different to the physiological flow provided by a beating heart. Natural aortic flow is triphasic in nature: through systolic, early diastolic and late diastolic phases. Traditional cardiac pumps do not replicate this flow.

The effects of pulsatile or steady pumping of blood rather than physiological flow during surgery are not well understood. It is at least possible that using a pump which more closely replicates patient physiology will lead to improved patient outcomes.

While mean blood pressure and blood flow rates can be matched using a pump providing steady flow, the dynamic energy of the blood flow is quite different between pulsatile and steady flow pumping. In particular, it is desirable to provide an appropriate degree of Surplus Hemodynamic Energy (SHE); that is, the difference between the Energy Equivalent Pressure delivered by a pulsatile pump with the mean arterial pressure. SHE can be an important factor in effect end-organ perfusion, with likely effect on capillary flow.

A further disadvantage of centrifugal cardiac pumps used in surgery is that pump heads are generally single-use items, as it is impractical to completely clean all blood residue from a pump. This represents a significant expense in cardiac surgery.

The present invention seeks to provide a cardiac pump which mimics a physiological blood flow, and is able to be safely reused.

The inventor of this application is also the inventor of the International Patent Application published under number WO2019/163520, the contents of which are incorporated herein by reference.

### SUMMARY OF THE INVENTION

According to one aspect of the present invention there is provided a pump having a fluid line formed from flexible tubing, the pump having a actuator, the actuator having an actuating member arranged to move between a first orientation and a second orientation such that the actuating member at least partially occludes the fluid line when in its second orientation; the actuator being arranged to move in a direction generally perpendicular to the fluid line, the pump being operable such that when the actuator moves from the first orientation to the second orientation it acts to peristaltically force fluid in the fluid line towards a fluid outlet.

The actuator may be a linear actuator having an actuating member which moves in a linear fashion between the first orientation and the second orientation.

Alternatively, the actuator may be a pivoting actuator having an actuating member arranged to pivot about a pivot axis between the first orientation and the second orientation. Preferably the pivot axis is parallel to the direction of the fluid line.

The pump can be programmed to match the physiological blood flow profile of a human patient or an animal.

The pump may have a control means arranged to control desired parameters of fluid flow. A first controllable parameter is preferably stroke volume, with a typical stroke volume between 5 ml and 100 ml. A second controllable parameter is preferably pulse rate, with a typical pulse between 60 and 120 beats per minute. A third controllable parameter may be the systolic:diastolic ratio, which controls the proportion of each actuating member stroke in a forward direction and a rearward direction. A fourth controllable parameter may be the acceleration rate of the actuating member.

Alternatively, the control means may include input for desired Surplus Hemodynamic Energy, and the pump may be calibrated to provide a desired SHE.

The first orientation may be free of the fluid line, such that the fluid line is not occluded. In a preferred embodiment, the first orientation represents a partial occlusion of the fluid line, with the second orientation representing a greater occlusion.

The pump preferably includes a check valve located at an inlet end of the fluid line. The check valve prevents backflow, ensuring that fluid flows in the desired direction along the fluid line.

Although the pump was developed for use as a cardiac pump during bypass procedures, it is considered to have other applications, both medical and industrial. In addition to cardiopulmonary bypass and extracorporeal membrane oxygenation procedures, other treatments are made possible by use of the present invention to supplement the perfusion of a human patient's or an animal's organ, mimicking the physiological flow profile of the artery that supplies oxygenated blood to the affected organ. This is done by diverting part of the oxygenated blood from the lower extremities to the intended organ.

According to one such aspect of the present invention there is provided a method for treating brain injury, dementia or stroke in a subject in need thereof, by diverting part of the oxygenated blood from lower extremities to provide more perfusion to brain cells using a programmable pump to synchronize the pulses with the patient's physiological blood flow in order to assist with healing of injured brain cells or to provide a treatment option for vertebrobasilar insufficiency.

According to another such aspect of the present invention there is provided a method for treating cancer patients by diverting oxygenated blood from lower extremities such as femoral arties to arteries of injured organs that have undergone cancer treatment/chemotherapy by using a programmable pump to synchronize the pulses with the patient's physiological blood flow in order to assist with healing of injured organs. Possible organs which can be treated include the kidneys, lungs, pancreas, stomach and liver.

According to yet another aspect of the present there is provided a method for treating chronic wounds by using a programmable pump synchronised with the patient's ECG pulse to supply adequate physiological flow of oxygenated blood to wound tissues near the wound site. It is anticipated that this method may be efficacious in assisting healing, particularly in diabetic patients.

### BRIEF DESCRIPTION OF THE DRAWINGS

It will be convenient to further describe the invention with reference to preferred embodiments of the present invention. Other embodiments are possible, and consequently the particularity of the following discussion is not to be understood as superseding the generality of the preceding description of the invention. In the drawings:

FIG. 1 is a perspective of a pump in accordance with a first embodiment of the present invention;

FIG. 2 is a schematic representation of an artificial heart incorporating a similar pump to that of FIG. 1;

FIG. 3 is a perspective of a pump in accordance with a second embodiment of the present invention;

FIG. 4 is a close up view of a portion of the pump of FIG. 3;

FIGS. 5 to 9 are echocardiograph charts representing different settings of the pumps of FIGS. 1 and 3;

FIG. 10 is a graphical representation of the operation of the pumps of FIGS. 1 and 3;

FIG. 11 is a picture of a control panel for use with the pumps of FIGS. 1 and 3; and

FIG. 12 is a graph demonstrating calibration of the pumps of FIGS. 1 and 3.

### DETAILED DESCRIPTION OF PREFERRED EMBODIMENTS

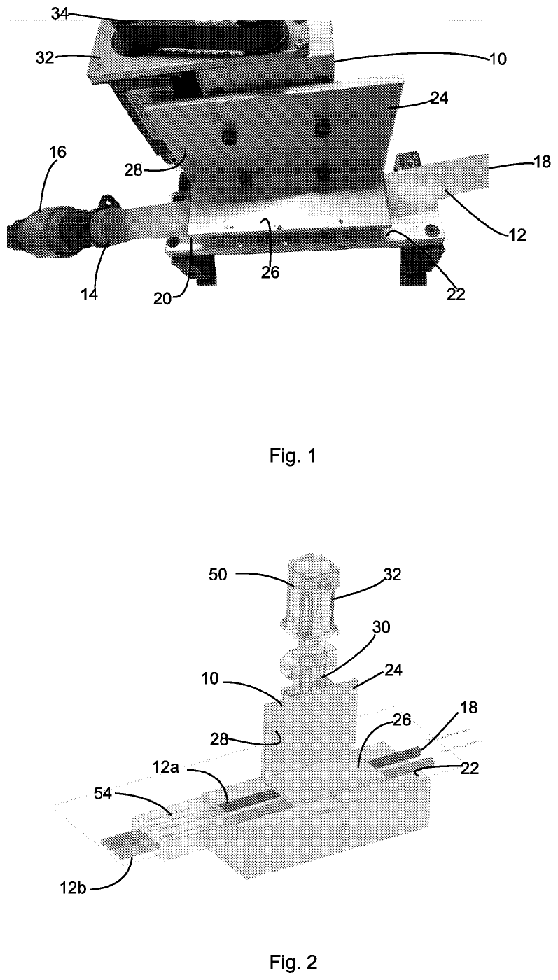

A first embodiment of the present invention, a pump 10, is shown in FIGS. 1 and 2. The pump 10 is arranged to force fluid (notionally blood) through a tube 12. In this embodiment the tube 12 is formed from platinum coated silicone tubing having an internal diameter of 19 mm. It is considered that bio-compatible tubing having an internal diameter between 6 mm and 25 mm may be suitable for this purpose.

The tube 12 has an inlet 14 associated with a check valve 16. The tube has an outlet 18. In between the tube inlet 14 and outlet 18 is an operating portion 20.

The operating portion 20 is arranged to rest against a rigid base plate 22. A generally L-shaped actuator 24 is positioned alongside the operating portion 20.

The actuator 24 has an actuating member being a compressing plate 26 which is generally horizontal, and parallel with the base plate 22. The actuator 24 has a supporting plate 28 which is perpendicular to the compressing plate 26. The supporting plate 28 is coupled to a linear slide 30 on a servo motor 32. The servo motor 32 is driven by a belt 34, and provides reciprocal linear motion to the linear slide 30 and thus to the actuator 24.

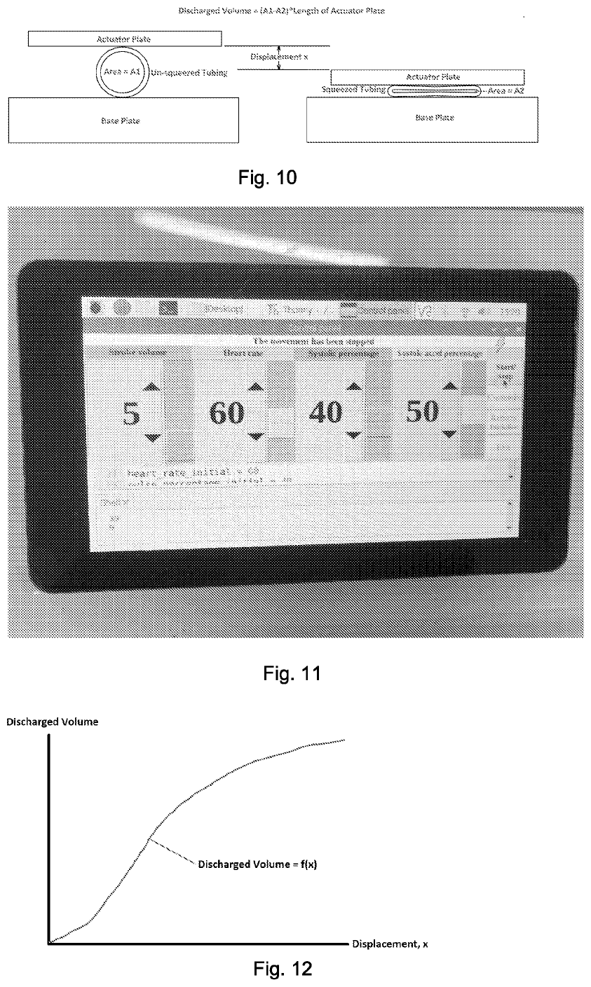

The servo motor 32 and actuator 24 may be calibrated such that the actuator reciprocates between a first position wherein the compressing plate 26 sits immediately adjacent the operating portion 20 of the tube 12, and a second position wherein the compressing plate 26 is moved towards the base plate 22, thus compressing the operating portion 20 between the compressing plate 26 and base plate 22. This is shown schematically in FIG. 10. It will be understood that this substantially occludes the tube 12, forcing fluid to flow towards the outlet 18. This generally mimics the systolic phase of a heart's action.

Calibration of the actuator allows for precise control of a blood flow profile. This is shown in FIG. 12.

Return of the actuator 24 to its first position allows the tube 12 to elastically return to a generally cylindrical shape. This removes the forcing action of the pump, creating a relative pressure drop which encourages the flow of fluid from the inlet. This generally mimics the diastolic phase of a heart's action.

It is anticipated that the closest alignment of pump performance with human physiology will have the actuator 24 partially occluding the tube 12 in its first position, and more fully occluding the tube 12 in its second position.

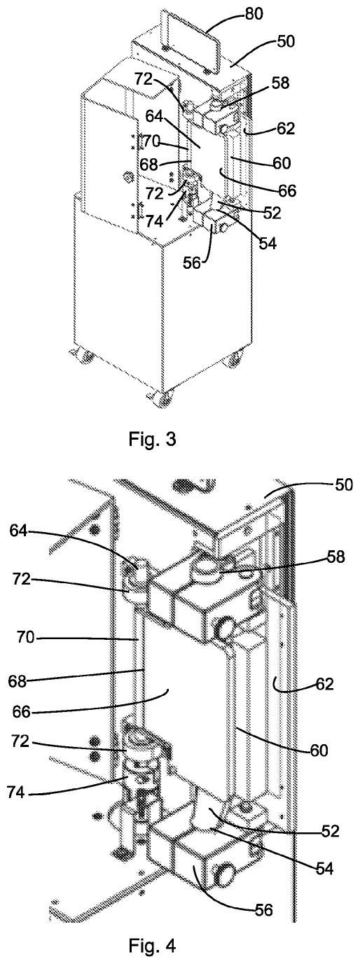

A second embodiment of the present invention, a pump 50, is shown in FIGS. 3 and 4. The pump 50 is arranged to force fluid (notionally blood) through a tube 52. The tube 52 is essentially the same as the tube 12, including an inlet 54 associated with a check valve 56, an outlet 58, and an operating portion 60.

The operating portion 60 is arranged to rest against a rigid base plate 62. An actuator 64 is positioned alongside the operating portion 60.

The actuator 64 has an actuating member being a compressing plate 66. The compressing plate 66 has a side edge 68 which is fixed to an axle 70. The axle 70 is parallel to the operating portion 60 of the tube 52.

The axle 70 is supported by upper and lower bearings 72. A drive motor 74 extends alongside the lower bearing 72. The drive motor 74 includes a cam mechanism (not shown) arranged to convert rotation of a drive shaft into back-and-forth pivoting of the axle 70. The pivoting of the axle 70 causes pivoting of the compressing plate 66 about the axle 70, between a first position wherein the compressing plate 66 sits immediately adjacent the operating portion 60 of the tube 52, and a second position wherein the compressing plate 66 is moved towards the base plate 62, thus compressing the operating portion 60 between the compressing plate 66 and base plate 62. The pump 50 thus has the same mimicking effect as the pump 10.

It will be appreciated that the use of appropriate gearing mechanisms such as planetary gears allow for high efficiency of the pump 50.

The pump 50 has a control panel 80 mounted thereto.

FIG. 11 shows a screen from a possible operating control panel 80, indicating four parameters which can be adjusted in order to best match a patient's actual echocardiology.

The first parameter to be controlled is pulse rate. This is simply set by the cycle time of the pump 10, 50; that is, the time between each ‘squeezing’ of the tube 12, 52. Typical pulse rates used in surgical procedures are expected to be between 40 and 120 ‘beats’ per minute.

The second parameter to be controlled is the stroke volume; that is, the volume of blood pumped during each cycle. This can be adjusted in a ‘micro’ sense by adjusting the length of each stroke of the pump 10, 50; that is, by adjusting the second position of the compressing plate 26, 66 to alter the degree of occlusion of the tube 12, 52. In a ‘macro’ sense large changes in stroke volume may require the changing of tubes 12, 52 to different tubes with larger or smaller diameters. Typical stroke volumes used in surgical procedures are expected to be between 0.5 litres per minute and 6.0 litres per minute.

The third parameter to be controlled is known as the systolic percentage. This is the percentage of the stroke cycle time when the compressing plate 26, 66 is moving towards the base plate 22, 62. It will be appreciated that the speed of movement for the compressing plate 26, 66 can be different depending on the direction in which it is moving. Typical systolic percentages used is surgical procedures are expected to be between 20% and 80%.

The fourth parameter to be controlled is known as the systolic acceleration percentage. It will be appreciated that the compressing plate 26, 56 need not move at a constant speed during occlusion of the tube 12, 52, and that adjusting the rate of acceleration will have an effect on the patient echocardiology.

FIG. 5 shows a typical patient echocardiology for a pump 10, 50 operating at 60 beats per minute with a flow rate of 5 litres per minute (lpm) and systolic percentage and systolic acceleration percentage both at 50%.

FIG. 6 shows a patient echocardiology for a pump 10, 50 operating at 60 beats per minute with a flow rate of 0.85 lpm, with the systolic percentage at 10%.

FIG. 6 shows a patient echocardiology for a pump 10, 50 operating at 20 beats per minute with a flow rate of 1.42 lpm, with the systolic percentage at 30%.

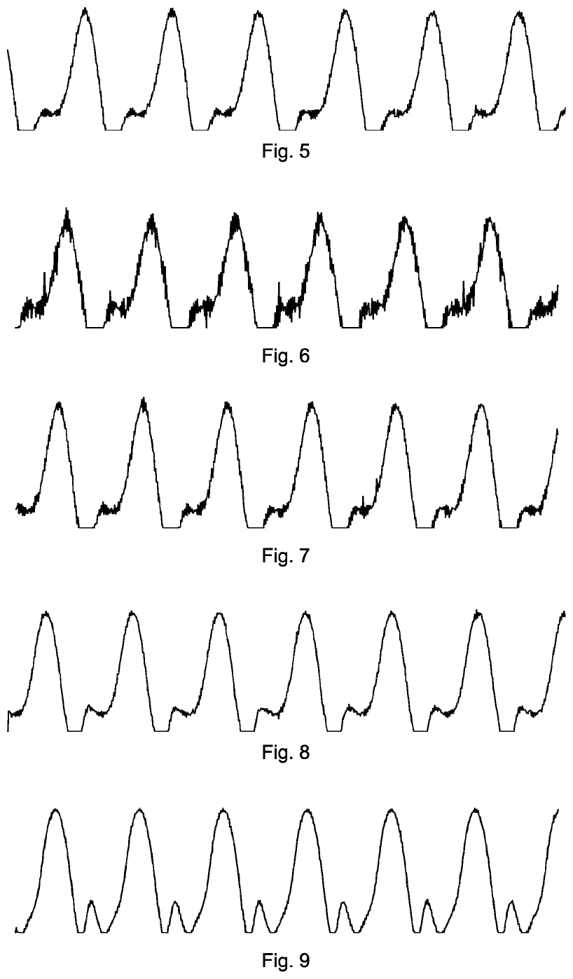

FIG. 7 shows a patient echocardiology for a pump 10, 50 operating at 60 beats per minute with a flow rate of 1.92 lpm, with the systolic percentage at 30%.

FIG. 8 shows a patient echocardiology for a pump 10, 50 operating at 60 beats per minute with a flow rate of 3.8 lpm, with the systolic percentage at 70%.

FIG. 9 shows a patient echocardiology for a pump 10, 50 operating at 60 beats per minute with a flow rate of 2.0 lpm, with the systolic percentage at 50% and the systolic acceleration at 80%.

It is anticipated that the pump may be used to mimic arterial flow in any artery, and potentially in multiple arteries simultaneously. It is proposed that a number of pumps may be used, with each pump providing physiological-style flow to particular arteries, rather than through a single access point. The anticipated advantages of this approach include:
a) The PSV and pressure of physiological flow at a canulla will be reduced significantly relative to a single access point, making it possible to implement true physiological flow.
b) More oxygenated blood can be directed to the brain and organs within the chest cavity to ensure adequate supply of oxygenated blood to the critical organs during open heart surgery, thus potentially preventing injuries and organ failures.
c) A longer period of surgery might be possible which will enable surgeons to fix more complicated clinical disease due to safer perfusion with distributed perfusion.

It is further proposed that an artificial heart 50 can be made from this new invention as shown in FIG. 2. First tubes 12a represent a Left Ventricle where oxygenated blood will be drawn from left and right pulmonary veins combined inside a manifold 54 and passing through a check valve during diastole. The actuator 24 pushes the first tubes 12a against the base plate 22 to pump oxygenated blood to the rest of the body through the ascending aorta during systole. Likewise second tubes 12b represent a Right Ventricle where de-oxygenated blood will be drawn from superior and inferior vena cavas; combined inside the manifold 54; and passed through a check valve during diastole. The actuator 24 pushes the second tubes 12b against the base plate 22 to pump the de-oxygenated blood to the lung through pulmonary artery during systole.

Modifications and variations as would be apparent to a skilled addressee are deemed to be within the scope of the present invention.

1. A pump having a fluid line formed from flexible tubing, the pump having a actuator, the actuator having an actuating member arranged to move between a first orientation and a second orientation such that the actuating member at least partially occludes the fluid line when in its second orientation; the actuator being arranged to move in a direction generally perpendicular to the fluid line, the pump being operable such that when the actuator moves from the first orientation to the second orientation it acts to peristaltically force fluid in the fluid line towards a fluid outlet.

2. A pump as claimed in claim 1, wherein the actuator is a linear actuator having an actuating member which moves in a linear fashion between the first orientation and the second orientation.

3. A pump as claimed in claim 1, wherein the actuator is a pivoting actuator having an actuating member arranged to pivot about a pivot axis between the first orientation and the second orientation.

4. A pump as claimed in claim 3, wherein the pivot axis is parallel to the direction of the fluid line.

5. A pump as claimed in claim 1, wherein the pump has a control means arranged to control desired parameters of fluid flow.

6. A pump as claimed in claim 5, wherein one controllable parameter is stroke volume.

7. A pump as claimed in claim 5, wherein one controllable parameter is pulse rate.

8. A pump as claimed in claim 5, wherein one controllable parameter is the systolic:diastolic ratio.

9. A pump as claimed in claim 5, wherein one controllable parameter is the acceleration rate of the actuating member.

10. A pump as claimed in claim 5, wherein one controllable parameter is desired Surplus Hemodynamic Energy.

11. A pump as claimed in claim 1, wherein the first orientation is free of the fluid line, such that the fluid line is not occluded.

12. A pump as claimed in claim 1, wherein the first orientation represents a partial occlusion of the fluid line, with the second orientation representing a greater occlusion.

13. A pump as claimed in claim 1, wherein the pump includes a check valve located at an inlet end of the fluid line.

14. A method for treating brain injury, dementia or stroke in a subject in need thereof, by diverting part of the oxygenated blood from lower extremities to provide more perfusion to brain cells using a programmable pump to synchronize the pulses with the patient's physiological blood flow in order to assist with healing of injured brain cells or to provide a treatment option for vertebrobasilar insufficiency.

15. A method for treating cancer patients by diverting oxygenated blood from lower extremities such as femoral arties to arteries of injured organs that have undergone cancer treatment/chemotherapy by using a programmable pump to synchronize the pulses with the patient's physiological blood flow in order to assist with healing of injured organs.

16. A method for treating chronic wounds by using a programmable pump synchronised with the patient's ECG pulse to supply adequate physiological flow of oxygenated blood to wound tissues near the wound site.
